(19) United States
(12) Patent Application Publication
(10) Pub. No.: US 2003/0124542 A1
(43) Pub. Date: Jul. 3, 2003

(54) Methods for mapping the chromosomal loci of genes expressed by a cell

(75) Inventors: Shishir Shah, Houston, TX (US); Mansoor Mohammed, Houston, TX (US)

Correspondence Address: GREGORY P. EINHORN, Fish & Richardson P.C., Suite 500, 4350 La Jolla Village Drive, San Diego, CA, 92122, US

(73) Assignee: Spectral Genomics, Inc., Houston, TX

(21) Appl. No.: 10/071,837

(22) Filed: Feb. 8, 2002

Related U.S. Application Data
(60) Provisional application No. 60/346,441, filed on Dec. 28, 2001.

(51) Int. Cl. C12Q 1/68

(52) U.S. Cl. 435/6

(57) ABSTRACT

The invention provides novel methods to map the location and measure the expression of some or all the genes expressed by a cell using array-based nucleic acid hybridizations. The use and applicability of the invention includes identification of tissue specific genes and identifying different tissue types during growth and development. The methods also can be used to identify genes expressing abnormal types of transcripts or abnormal levels or transcripts. Thus, the invention can be used to identify genes encoding transcripts associated with disease processes, such as cancer.

### CROSS-REFERENCE TO RELATED APPLICATION

The present application claims the benefit of priority under 35 U.S.C. §119(e) of U.S. Provisional Application No. 60/346,441, filed Dec. 28, 2001 (attorney docket number 13320-010P01). The aforementioned application is explicitly incorporated herein by reference in its entirety and for all purposes.

### TECHNICAL FIELD

This invention relates to molecular biology, genetic diagnostics and array, or “biochip,” technology. In particular, the invention provides methods using nucleic acid arrays to map, or identity, the chromosomal loci encoding all or some of the transcripts of a cell or tissue. The use and applicability of the invention includes identification of tissue specific genes and identifying different tissue types during growth and development. The methods also can be used to identify genes expressing abnormal types of transcripts or abnormal levels or transcripts. Thus, the invention can be used to identify genes encoding transcripts associated with disease processes, such as cancer.

### BACKGROUND

Disturbances in a cell's cycle and function may be a cause of a disease state. These disturbances may be due to any number of etiological factors. One possible etiological factor may be a perturbation in a cell's mRNA expression profile. This may directly reflect a fluctuation in the cell's genomic DNA sequence copy number. Alternatively, subtle mutations or chromosomal rearrangements which do not affect the cell's genomic DNA sequence copy number may also result in perturbations of the cell's mRNA expression profile, or transcriptome. Methods of mapping a transcribed gene to a specific location on a chromosome are slow and labor intensive. A rapid means for identifying a chromosomal loci of a gene expressed by a cell would be an efficient tool to uncover the primary causes of many disease states.

### SUMMARY

The invention provides an array-based method for identifying a chromosomal loci of a gene expressed by a cell, the method comprising: (a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; (b) providing a sample comprising a nucleic acid sequence comprising a detectable label, wherein the nucleic acid sequence comprises the same sequence or a sequence complementary to a sequence of a transcript expressed by a cell; (c) contacting the sample of step (b) with the array of step (a) under conditions wherein the labeled nucleic acid can specifically hybridize to the genomic nucleic acid segments immobilized on the array; and, (d) identifying to which discrete and known spots on the substrate surface are specifically hybridized to a labeled nucleic acid segment, wherein the identification is made by detecting a specifically hybridized nucleic acid sequence, thereby identifying a chromosomal loci of a gene expressed by a cell. The invention provides an array-based method for identifying a chromosomal loci of a gene expressed by a cell.

In one aspect, the labeled nucleic acid sequence comprises the same sequence or a sequence complementary to a subset of transcripts expressed by the cell. The labeled nucleic acid sequences can comprise the same sequence or a sequence complementary to all of the transcripts expressed by the cell, or, all of the transcripts expressed by a tissue.

In alternative aspects, the transcript or the sequence complementary to the transcript comprises a HER2/neu or a Neu/ErbB2 transcript sequence. The transcript or the sequence complementary to the transcript can comprise a c-fos, a c-fas or a c-jun transcript sequence. The transcript or the sequence complementary to the transcript can comprise a DAX-1 or a DAX-2 transcript sequence.

The invention provides an array-based method for identifying a chromosomal loci of a gene differentially expressed by different cells or by a cell under different conditions by performing an array-based comparative genomic hybridization (CGH), comprising the following steps: (a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; (b) providing a first sample, wherein the sample comprises a plurality of nucleic acid sequences comprising a first detectable label and the nucleic acid sequence comprises the same sequence or a sequence complementary to all of the transcripts or a subset of transcripts expressed by a first cell; (c) providing a second sample, wherein the sample comprises a plurality of nucleic acid sequences comprising a second detectable label, wherein the nucleic acid sequence comprises the same sequence or a sequence complementary to all or a subset of the transcripts expressed by a second cell; (d) contacting the samples of step (b) and step (c) with the array of step (a) under conditions wherein the nucleic acid in the samples can specifically hybridize to the immobilized nucleic acid of step (a); and (e) identifying to which discrete and known spots on the substrate surface are specifically hybridized to a nucleic acid sequence of step (b) and identifying to which discrete and known spots on the substrate surface are specifically hybridized to a nucleic acid sequence of step (c), wherein the identification is made by detecting a specifically hybridized first and a specifically hybridized second sample nucleic acid sequence, thereby identifying a chromosomal loci of a gene differentially expressed by a first cell compared to a second cell.

In one aspect, step (e) further comprises measuring the amount of specifically hybridized first and second label on each discrete and known spot and comparing the amount of first and second sample nucleic acid sequence on each discrete and known spot, thereby determining the level of expression of the differentially expressed gene in the first cell as compared to the second cell.

In one aspect, the first cell is a normal cell and the second cell has an abnormal phenotype or a disease phenotype. The abnormal phenotype can comprise a neoplastic or hyperplastic phenotype. The neoplastic phenotype can be any cancer, e.g., a breast cancer, a bone cancer and the like. In one aspect, the first cell is a normal cell and the second cell has phenotype of an injured cell. In one aspect, the first cell is a normal cell and the second cell has an altered phenotype because it has been exposed to an environmental stress. The environmental stress can comprise a high or a low or a change in temperature, a change in salinity or extracellular ion concentration or pH. In one aspect, the environmental stress comprises an exposure to a chemical. The chemical can be a carcinogen, such as a drug, a medicine, an irradiation, e.g., an X-ray or radiation from an isotope.

In one aspect, the first cell is a cell in one state and the second cell is the same or similar cell in a different state. For example, the first cell can be an unstimulated cell and the second cell can be the unstimulated cell after stimulation. The first cell can be an undifferentiated cell and the second cell can be the undifferentiated cell after stimulation.

In alternative aspects, the nucleic acid sequence comprises an RNA (e.g., an mRNA), a DNA, a cDNA or an expressed sequence tag (EST).

The nucleic acid sequence can be complementary to a transcript comprising a sequence representative of the full length of the transcript. In alternative aspects, the nucleic acid sequence complementary to a transcript is between about 12 to about 500 bases in length, between about 25 to about 250 bases in length, between about 50 to about 150 bases in length, or between about 100 to about 125 bases in length.

The cloned genomic nucleic acid segment can be cloned in a construct comprising an artificial chromosome. In alternative aspects, the artificial chromosome comprises a bacterial artificial chromosome (BAC), a human artificial chromosome (HAC) a yeast artificial chromosome (YAC), a transformation-competent artificial chromosome (TAC) or a bacteriophage P1-derived artificial chromosome (PAC). The cloned nucleic acid segment can be cloned in a construct comprising a cosmid vector, a plasmid vector or a viral vector.

The cloned nucleic acid segment can be between about 50 kilobases (0.5 megabase) to about 500 kilobases (5 megabases) in length, between about 100 kilobases (1 megabase) to about 400 kilobases (4 megabases) in length, or, is about 300 kilobases (3 megabases) in length.

In alternative aspects, the cell expressing the transcript comprises a body fluid sample, a cell sample or a tissue sample. The cell expressing the transcript can comprise a cancer cell or a tumor cell sample. The cell expressing the transcript can comprise a biopsy sample, a blood sample, a urine sample, a cerebral spinal fluid (CSF) sample, an amniotic fluid sample, a chorionic villus sample, an embryonic cell or embryo tissue sample. The cell expressing the transcript can comprise a mammalian cell, such as a human cell.

In one aspect, the methods of the invention further comprise a washing step, wherein nucleic acid in the sample not specifically hybridized to the genomic nucleic acid segments immobilized on the array are removed. The washing step can comprise use of a solution comprising a salt concentration of about 0.02 molar at pH 7 at a temperature of at least about 50° C. The washing step can comprise use of a solution comprising a salt concentration of about 0.15 M at a temperature of at least about 72° C. for about 15 minutes. The washing step can comprise use of a solution comprising a salt concentration of about 0.2×SSC at a temperature of at least about 50° C. for at least about 15 minutes.

The invention comprises a kit comprising the following components: (a) an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; and, (b) instructions for using the array for identifying a chromosomal loci of a gene expressed by a cell as set forth in the methods of the invention. The kit can further comprise materials to prepare a sample comprising a genomic nucleic acid for application to the array. The kit can further comprise materials to isolate, clone or amplify the transcripts of a cell. The kit can further comprise materials to prepare a cDNA library. The kit can further comprise materials to label sample. The kit can further comprise an array-immobilized nucleic acid, e.g., in the form of a biochip, or array. The kit can further comprise a sample of wild type, or normal, nucleic acid. The kit can further comprise wild type, or normal, nucleic acid. In one aspect, the nucleic acid is labeled. The wild type, or normal, nucleic acid comprises a human wild type genomic nucleic acid. In alternative aspects, the arrays of the kit comprise a G-CHIP™, a SPECTRALCHIP™ Mouse BAC Array or a SPECTRALCHIP™ Human BAC Array.

The invention provides an array-based method for identifying a gene expressed in a tissue-specific or a cell-specific manner, the method comprising: (a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; (b) providing a sample comprising a nucleic acid sequence comprising a detectable label, wherein the nucleic acid sequence comprises the same sequence or a sequence complementary to a sequence of a transcript expressed by a cell and expression of the transcript is cell-specific or tissue specific; (c) contacting the sample of step (b) with the array of step (a) under conditions wherein the labeled nucleic acid can specifically hybridize to the genomic nucleic acid segments immobilized on the array; and, (d) identifying to which discrete and known spots on the substrate surface are specifically hybridized to a labeled nucleic acid segment, wherein the identification is made by detecting a specifically hybridized nucleic acid sequence, thereby identifying a chromosomal loci of a tissue-specific or a cell-specific gene.

The invention provides an array-based method for identifying a gene specifically expressed in a tissue or a cell type during a specific growth or developmental process, the method comprising: (a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; (b) providing a sample comprising a nucleic acid sequence comprising a detectable label, wherein the nucleic acid sequence comprises the same sequence or a sequence complementary to a sequence of a transcript expressed by a cell and expression of the transcript is specific for a tissue or a cell type during a specific growth or developmental process; (c) contacting the sample of step (b) with the array of step (a) under conditions wherein the labeled nucleic acid can specifically hybridize to the genomic nucleic acid segments immobilized on the array; and, (d) identifying to which discrete and known spots on the substrate surface are specifically hybridized to a labeled nucleic acid segment, wherein the identification is made by detecting a specifically hybridized nucleic acid sequence, thereby identifying a chromosomal loci of a gene specifically expressed in a growth or a developmental process. Thus, the methods of the invention can be used to identify which genes will be differentially expressed in different developmental and growth processes. Similarly, by identifying which genes are expressed, the methods of the invention can be used to identify which developmental or growth process a cell is in. For example, the methods of the invention can be used to identify if a cell is to be or is, e.g., a nerve cell, a brain cell, a heart tissue cell, a blood cell and the like.

Similarly, by identifying which genes are expressed, the methods of the invention can be used to identify the sex of a cell or can be used to track a phenotypic sex change in a cell or a tissue.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits cited herein are hereby expressly incorporated by reference for all purposes.

### DETAILED DESCRIPTION

The invention provides novel methods to map, or identify, the location and measure the expression of some or all the genes expressed by a cell. In one aspect, the methods of the invention comprise performing comparative genomic hybridization (CGH) of test and reference sample cDNA populations. Each population can be representative of a subpopulation or all the transcripts of a cell (the total mRNA of a cell).

The methods comprise use of array-based nucleic acid hybridizations. The arrays (biochips) comprise immobilized genomic clones. In alternative aspects, the clones on the chip represent a defined subset or substantially the entire genomic content of a cell.

In addition to mapping the loci of genes expressing transcripts, the methods of the invention can be used to assess the levels of expression of any or all of a cell's genes. For example, the methods of the invention can map and measure genes essential for the survival of the cell, such as housekeeping genes. These methods of the invention can be used to map and measure the expression of genes required for any specific function of the cell, such as the expression of particular cytokines by various cells of the immune system or melanin by melanocytes. Thus, the methods of the invention provide a novel way to perform gene mapping and expression studies.

The methods of the invention comprise making and using nucleic acids having the same sequence or a sequence complementary to a sequence of a transcript (i.e., an mRNA or message) expressed by a cell. These sequences can be reverse transcription (RT) reaction products (cDNA made from RNA) or nucleic acids amplified or otherwise made from these products, e.g., by a DNA polymerase reaction or a de novo synthetic synthesis.

In one aspect, the methods comprise mapping, or identifying, a chromosomal loci of a gene or genes expressing one or more transcripts in a cell. For example, genes whose expression is associated with a hyperplastic, cancerous or neoplastic phenotype can be mapped and the levels of transcript assessed. In alternative aspects, HER2/neu, Neu/ErbB2, c-fos, c-fas or c-jun genes are mapped.

In one aspect, the methods comprise mapping, or identifying, a chromosomal loci of a gene differentially expressed by different cells or by a cell under different conditions by performing an array-based comparative genomic hybridization (CGH) (the relative levels of transcripts in two samples can also be assessed using CGH). For example, as described in Example 3, below, transcripts can be from the germinal ridge of mice at the same stage of development where one sample is from “normal” mice and a second sample from mice that demonstrate phenotypic sex-reversal. In this example, genes responsible for phenotypic sex-reversal can be mapped, e.g., DAX-1 or DAX-2 genes. The CGH methods of the invention can identify and compare transcripts and the genes that encode them from a cell or cells having a “normal,” “wild type,” “reference” or “baseline” phenotype with those from a cell or cells having a changed, differentiated, activated, abnormal, pathologic, a typical or altered phenotype. For example, the transcripts expressed by a cell or cell population in one state can be compared to the transcripts expressed by the same cell or cell population in a different state. The different state can be, e.g., an activated or a differentiated state. The different state can be, e.g., an infected or a pathologic state. The first and the second (different) state can be, e.g., before and after exposure to a chemical or an environmental stimulus, such as a change in temperature, pH, osmolarity and the like. The chemical can be a hormone, a cytokine, a lymphokine, an antibody and the like.

Total mRNA extracted from a cell reflects the genes expressed by that cell. Thus, in practicing the methods of the invention, all or some of the transcripts of a cell are converted into DNA using, e.g., reverse transcriptase polymerase chain reaction (RT-PCR) or an equivalent amplification reaction. RT-PCR allows the mRNA extracted from a cell to be transcribed into what is termed cDNA, or DNA that is homologous to the exonic regions of the genes that were expressed by the cell. Therefore, the hybridization of cDNAs to arrays comprising genomic DNA clones that span the entire genome (e.g., G-CHIPS™, Spectral Genomics, Houston Tex.), will allow the detection of the chromosomal loci actively expressed by the cells. Moreover, if the cDNA from a control population of cells is co-hybridized with the cDNA from an equivalent tumorigenically or developmentally altered cell population, not only would the fluctuations in the cDNA profiles be determined but also a direct co-relation to chromosomal loci on a whole genome basis. Combined with the on-going genome project, this approach represents a significant advancement in our ability to directly determine the chromosomal loci and gene sequences that may be correlated to etiological consequences in the onset of neoplasia or developmental abnormalities.

Also provided are kits comprising instructions to use the methods of the invention. The kits can include instructions for practicing the methods of the invention and an array to practice the invention, e.g., a SPECTRALCHIP™ Mouse BAC array or a SPECTRALCHIP™ Human BAC array. The kits can also include, for the convenience of the practitioner, materials for extracting RNA from a sample (such as a cell extract or tissue sample) and preparing, e.g., cDNA or EST libraries. The kits can also include materials for labeling of nucleic acid to be applied to the array. In one aspect, the kits can also include labeled “wild type” cDNA or ESTs of a particular cell type, e.g., cDNA or EST nucleic acid having the same sequence as transcripts of genomes not known to have any or substantially having no chromosomal abnormalities and/or any contiguous gene abnormalities. The “wild type” nucleic acid can comprise a substantially complete transcriptome, e.g., nucleic acid the same or complementary to all of the transcripts of a cell. This may be useful if the practitioner will be performing a comparative genomic hybridization (CGH).

### Definitions

Unless defined otherwise, all technical and scientific terms used herein have the meaning commonly understood by a person skilled in the art to which this invention belongs. As used herein, the following terms have the meanings ascribed to them unless specified otherwise.

The terms “array” or “microarray” or “DNA array” or “nucleic acid array” or “chip” or “biochip” as used herein is a plurality of target elements, each target element comprising a defined amount of one or more biological molecules, e.g., genomic nucleic acid segments, immobilized on a defined location on a substrate surface; as described in further detail, below.

The term “aryl-substituted 4,4-difluoro-4-bora-3a, 4a-diaza-s-indacene dye” as used herein includes all “boron dipyrromethene difluoride fluorophore” or “BODIPY” dyes and “dipyrrometheneboron difluoride dyes” (see, e.g., U.S. Pat. No. 4,774,339), or equivalents, are a class of fluorescent dyes commonly used to label nucleic acids for their detection when used in hybridization reactions; see, e.g., Chen (2000) J. Org Chem. 65:2900-2906: Chen (2000) J. Biochem. Biophys. Methods 42:137-151. See also U.S. Pat. Nos. 6,060,324; 5,994,063; 5,614,386; 5,248,782; 5,227,487; 5,187,288.

The terms “cyanine 5” or “Cy5™” and “cyanine 3” or “Cy3™” refer to fluorescent cyanine dyes produced by Amersham Pharmacia Biotech (Piscataway, N.J.) (Amersham Life Sciences, Arlington Heights, Ill.), as described in detail, below, or equivalents. See U.S. Pat. Nos. 6,027,709; 5,714,386; 5,268,486; 5,151,507; 5,047,519. These dyes are typically incorporated into nucleic acids in the form of 5-amino-propargyl-2′-deoxycytidine 5′-triphosphate coupled to Cy5™ or Cy3™.

The terms “fluorescent dye” and “fluorescent label” as used herein includes all known fluors, including rhodamine dyes (e.g., tetramethylrhodamine, dibenzorhodamine, see, e.g., U.S. Pat. No. 6,051,719); fluorescein dyes; “BODIPY” dyes and equivalents (e.g., dipyrrometheneboron difluoride dyes, see, e.g., U.S. Pat. No. 5,274,113); derivatives of 1-[isoindolyl]methylene-isoindole (see, e.g., U.S. Pat. No. 5,433,896); and all equivalents. See also U.S. Pat. Nos. 6,028,190; 5,188,934.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which one nucleic acid will hybridize preferentially to second sequence (e.g., a sample genomic nucleic acid hybridizing to an immobilized nucleic acid probe in an array), and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions as used herein can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Those of ordinary skill will readily recognize that alternative but comparable hybridization and wash conditions can be utilized to provide conditions of similar stringency.

However, the selection of a hybridization format is not critical, as is known in the art, it is the stringency of the wash conditions that set forth the conditions which determine whether a soluble, sample nucleic acid will specifically hybridize to an immobilized nucleic acid. Wash conditions can include, e.g.: a salt concentration of about 0.02 molar at pH 7 and a temperature of at least about 50° C. or about 55° C. to about 60° C.; or, a salt concentration of about 0.15 M NaCl and a temperature of at least about 72° C. for at least about 15 minutes; or, a salt concentration of about 0.2×SSC at a temperature of at least about 50° C. or about 55° C. to about 60° C. for at least about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68oC for 15 minutes; or, equivalent conditions. Stringent conditions for washing can also be, e.g., 0.2×SSC/0.1% SDS at 420C. See Sambrook, Ausubel, or Tijssen (cited herein) for detailed descriptions of equivalent hybridization and wash conditions and for reagents and buffers, e.g., SSC buffers and equivalent reagents and conditions.

The phrase “nucleic acid sequence comprising a detectable label” or “nucleic acid sequence labeled with a detectable moiety” as used herein refers to a nucleic acid comprising a detectable composition, i.e., a label. The label can also be another biological molecule, as a nucleic acid, e.g., a nucleic acid in the form of a stem-loop structure as a “molecular beacon,” as described below. This includes incorporation of labeled bases (or, bases which can bind to a detectable label) into the nucleic acid by, e.g., nick translation, random primer extension, amplification with degenerate primers, and the like. The label can be detectable by any means, e.g., visual, spectroscopic, photochemical, biochemical, immunochemical, physical or chemical means. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin.

The term “nucleic acid” as used herein refers to a deoxyribonucleotide or ribonucleotide in either single- or double-stranded form. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

The term “genomic DNA” or “genomic nucleic acid” includes nucleic acid isolated from a nucleus of one or more cells, and, includes nucleic acid derived from (e.g., isolated from, amplified from, cloned from, synthetic versions of) genomic DNA. The genomic DNA can be from any source, as discussed in detail, below. The term “wild type genomic nucleic acid” means a sample of genomic nucleic acid having no known or substantially no known contiguous gene abnormalities.

The term “a sample comprising a nucleic acid” or “sample of nucleic acid” as used herein refers to a sample comprising a DNA or an RNA, or nucleic acid representative of DNA or RNA isolated from a natural source, in a form suitable for hybridization (e.g., as a soluble aqueous solution) to another nucleic acid or polypeptide or combination thereof (e.g., immobilized probes). The nucleic acid may be isolated, cloned or amplified; it may be, e.g., genomic DNA, episomal DNA, mitochondrial DNA, mRNA, or cDNA; it may be a genomic segment that includes, e.g., particular promoters, enhancers, coding sequences, and the like; it may also include restriction fragments, cDNA libraries or fragments thereof, etc. The nucleic acid sample may be extracted from particular cells, tissues or body fluids, or, can be from cell cultures, including cell lines, or from preserved tissue sample, as described in detail, below.

The term “HER-2/neu” refers to an oncoprotein frequently over-expressed in cancers because of overexpression of a HER-2/neu encoding gene or by gene amplification of a HER-2/neu encoding gene. See, e.g., Safran (2001) Am. J. Clin. Oncol. 24(5):496-949.

The term “erbB” refers to any member of the erbB family of receptor tyrosine kinases. The ErbB2 receptor tyrosine kinase (RTK) has been intensely pursued as a cancer therapy target due to its association with breast cancer and other neoplasias. See, e.g., Zhou (2001) Oncogene 20(42):6009-6017.

The terms “c-fos,” “c-fas” and “c-jun” refer well-characterized genes that encode oncogenic proteins. See, e.g., U.S. Pat. Nos. 5,888,764; 5,985,558; 6,103,890.

The term “DAX-1,” or, dosage-sensitive sex reversal, adrenal hypoplasia congenita (AHC) critical region on the X chromosome, gene 1, refers to an orphan nuclear receptor that represses transcription by steroidogenic factor-1 (SF-1), a factor that regulates expression of multiple steroidogenic enzymes and other genes involved in reproduction. The DAX-1 gene has been involved in the dosage sensitive sex reversal (DSS) phenotype, a male-to-female sex-reversal syndrome due to the duplication of a small region of human chromosome Xp21. Mutations in the human DAX1 gene (also known as AHC) cause the X-linked syndrome AHC, a disorder that is associated with hypogonadotropic hypogonadism. See, e.g., Wang (2001) Proc. Natl. Acad. Sci. USA 98:7988-7993; Goodfellow (2001) EXS 91:57-69.

### Generating and Manipulating Nucleic Acids

Practicing the methods and making and using the arrays used in the methods of the invention may involve the isolation, synthesis, cloning, amplification, labeling and hybridization (e.g., CGH) of nucleic acids. The nucleic acid for analysis comprises the same sequence or a sequence complementary to a sequence of one or more (or all) transcripts expressed by a cell. The immobilized nucleic acid on the arrays used in the methods of the invention is representative of genomic DNA, including defined parts of, or entire, chromosomes, or entire genomes. Comparative genomic hybridization (CGH) reactions, see, e.g., U.S. Pat. Nos. 5,830,645; 5,976,790, are discussed in further detail, below. Nucleic acid samples, and, in some aspects, immobilized nucleic acids, can be labeled with a detectable moiety, e.g., a fluorescent dye(s) or equivalent. For example, a first sample can be labeled with a fluor and a second sample labeled with a second dye (e.g., Cy3™ and Cy5™). In one aspect, each sample nucleic acid is labeled with at least one different detectable moiety, e.g., different fluorescent dyes, than those used to label the other samples of nucleic acids.

In some cases, the nucleic acids may be amplified using standard techniques such as PCR. Amplification can also be used to generate nucleic acid for hybridization to arrays, to subclone or to label a nucleic acid prior to hybridization. The sample and/or the immobilized nucleic acid can be labeled, as described herein. The nucleic acid for analysis comprising the same sequence or a sequence complementary to a sequence of one or more (or all) transcripts expressed by a cell can be a cDNA library, an EST or a transcript, or any nucleic acid or fragment generated therefrom. The probe on the array can be produced from and collectively can be representative of a source of nucleic acids from one or more particular (pre-selected) portions of, e.g., a collection of polymerase chain reaction (PCR) amplification products, substantially an entire chromosome or a chromosome fragment, or substantially an entire genome, e.g., as a collection of clones, e.g., BACs, PACs, YACs, and the like (see below). The array-immobilized nucleic acid or genomic nucleic acid sample may be processed in some manner, e.g., by blocking or removal of repetitive nucleic acids or by enrichment with selected nucleic acids.

Samples are applied to the immobilized probes (e.g., on the array) and, after hybridization and washing, the location (e.g., spots on the array) and amount of each dye are read. The array-immobilized nucleic acid can be in the form of cloned DNA, e.g., YACs, BACs, PACs, and the like, as described herein. As is typical of array technology, in one aspect, each “spot” on the array has a known sequence, e.g., a known segment of genome or other sequence. The invention can be practiced in conjunction with any method or protocol or device known in the art, which are well described in the scientific and patent literature.

General Techniques

The nucleic acids used to practice this invention, whether RNA, cDNA, ESTs, genomic DNA, vectors, viruses or hybrids thereof, may be isolated from a variety of sources, genetically engineered, amplified, and/or expressed/generated recombinantly. Any recombinant expression system can be used, including, in addition to bacterial cells, e.g., mammalian, yeast, insect or plant cell expression systems.

Alternatively, these nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization, G-banding, CGH, SKY, FISH and the like are well described in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Cloning of Genomic Nucleic Acids

Genomic nucleic acids used in the arrays and methods of the invention, e.g., those immobilized onto arrays, can be obtained and manipulated by cloning into various vehicles. If necessary, genomic nucleic acid samples can be screened and re-cloned or amplified from any source of genomic DNA. Thus, in various aspects, forms of genomic nucleic acid used in the methods of the invention (including arrays) include genomic DNA, e.g., genomic libraries, contained in mammalian and human artificial chromosomes, satellite artificial chromosomes, yeast artificial chromosomes, bacterial artificial chromosomes, P1 artificial chromosomes, recombinant vectors and viruses, plasmids, and the like.

Mammalian artificial chromosomes (MACs) and human artificial chromosomes (HAC) are, e.g., described in Ascenzioni (1997) Cancer Lett. 118:135-142; Kuroiwa (2000) Nat. Biotechnol. 18:1086-1090; U.S. Pat. Nos. 5,288,625; 5,721,118; 6,025,155; 6,077,697). MACs can contain inserts larger than 400 kilobase (Kb), see, e.g., Mejia (2001) Am. J. Hum. Genet. 69:315-326. Auriche (2001) EMBO Rep. 2:102-107, has built a human minichromosomes having a size of 5.5 kilobase.

Satellite artificial chromosomes, or, satellite DNA-based artificial chromosomes (SATACs), are, e.g., described in Warburton (1997) Nature 386:553-555; Roush (1997) Science 276:38-39; Rosenfeld (1997) Nat. Genet. 15:333-335). SATACs can be made by induced de novo chromosome formation in cells of different mammalian species; see, e.g., Hadlaczky (2001) Curr. Opin. Mol. Ther. 3:125-132; Csonka (2000) J. Cell Sci. 113 (Pt 18):3207-3216.

Yeast artificial chromosomes (YACs) can also be used and typically contain inserts ranging in size from 80 to 700 kb. YACs have been used for many years for the stable propagation of genomic fragments of up to one million base pairs in size; see, e.g., U.S. Pat. Nos. 5,776,745; 5,981,175; Feingold (1990) Proc. Natl. Acad. Sci. USA 87:8637-8641; Tucker (1997) Gene 199:25-30; Adam (1997) Plant J. 11:1349-1358; Zeschnigk (1999) Nucleic Acids Res. 27:21.

Bacterial artificial chromosomes (BACs) are vectors that can contain 120 Kb or greater inserts, see, e.g., U.S. Pat. Nos. 5,874,259; 6,277,621; 6,183,957. BACs are based on the E. coli F factor plasmid system and simple to manipulate and purify in microgram quantities. Because BAC plasmids are kept at one to two copies per cell, the problems of rearrangement observed with YACs, which can also be employed in the present methods, are eliminated; see, e.g., Asakawa (1997) Gene 69-79; Cao (1999) Genome Res. 9:763-774.

P1 artificial chromosomes (PACs), bacteriophage P1-derived vectors are, e.g., described in Woon (1998) Genomics 50:306-316; Boren (1996) Genome Res. 6:1123-1130; Ioannou (1994) Nature Genet. 6:84-89; Reid (1997) Genomics 43:366-375; Nothwang (1997) Genomics 41:370-378; Kern (1997) Biotechniques 23:120-124). P1 is a bacteriophage that infects E. coli that can contain 75 to 100 Kb DNA inserts (see, e.g., Mejia (1997) Genome Res 7:179-186; Ioannou (1994) Nat Genet 6:84-89). PACs are screened in much the same way as lambda libraries. See also Ashworth (1995) Analytical Biochem. 224:564-571; Gingrich (1996) Genomics 32:65-74.

Other cloning vehicles can also be used, for example, recombinant viruses; cosmids, plasmids or cDNAs; see, e.g., U.S. Pat. Nos. 5,501,979; 5,288,641; 5,266,489.

These vectors can include marker genes, such as, e.g., luciferase and green fluorescent protein genes (see, e.g., Baker (1997) Nucleic Acids Res 25:1950-1956). Sequences, inserts, clones, vectors and the like can be isolated from natural sources, obtained from such sources as ATCC or GenBank libraries or commercial sources, or prepared by synthetic or recombinant methods.

Amplification of Nucleic Acids

Amplification using oligonucleotide primers can be used to generate or manipulate, e.g., to subclone or generate fragments of, transcripts, cDNAs, ESTs, genomic nucleic acids used in the arrays, to incorporate label into immobilized or sample nucleic acids, to detect or measure levels of nucleic acids hybridized to an array, and the like. Amplification, typically with degenerate primers, is also useful for incorporating detectable probes (e.g., Cy5™- or Cy3™-cytosine conjugates) into nucleic acids representative of test or control genomic DNA to be used to hybridize to immobilized genomic DNA. Amplification can be used to quantify the amount of nucleic acid is in a sample, see, e.g., U.S. Pat. No. 6,294,338. The skilled artisan can select and design suitable oligonucleotide amplification primers. Amplification methods are also well known in the art, and include, e.g., polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques, e.g., nucleic acid sequence based amplification, or, “NASBA,” see, e.g., Birch (2001) Lett. Appl. Microbiol. 33:296-301; Greijer (2001) J. Virol. Methods 96:133-147. See also Berger (1987) Methods Enzymol. 152:307-316; Sambrook; Ausubel; U.S. Pat. Nos. 4,683,195 and 4,683,202.

Generating Sequences Representative of Transcripts

The invention comprises providing nucleic acid sequences comprising the same sequence or a sequence complementary to a sequence of a transcript expressed by a cell. The nucleic acid sequences can be representative of full-length transcripts, or fragments of the transcripts. The nucleic acid sequences can be generated by reverse transcriptase generation of a cDNA or EST library. Making and using cDNAs and ESTs and libraries therefrom are well known in the art. See, e.g., Yu (2001) Genome Res. 11:1392-1403; Neto (1997) Gene 186:135-142. See also, U.S. Pat. Nos. 6,265,165; 6,143,528; 6,136,569; 6,187,544; 6,114,154; 5,891,637; 5,837,468; 5,759,820.

If less than a full length cDNA or transcript is used, methods for selecting or making subsequences that selectively and specifically hybridize to a complementary sequence, e.g., an immobilized nucleic acid probe on an array, can be determined by paradigms well known in the art, see, e.g., Toschi (2000) Methods 22:261-269; Walton (1999) Biotechnol. Bioeng. 65:1-9. See also, U.S. Pat. Nos. 5,747,248; 6,171,820; 6,174,673; 6,323,030.

Hybridizing Nucleic Acids

In practicing the methods of the invention, samples of nucleic acid, e.g., isolated, reverse transcribed, cloned or amplified transcripts, are hybridized to immobilized nucleic acids. In alternative aspects, the hybridization and/or wash conditions are carried out under moderate to stringent conditions. An extensive guide to the hybridization of nucleic acids is found in, e.g., Sambrook Ausubel, Tijssen. Generally, highly stringent hybridization and wash conditions are selected to be about 5oC lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. Exemplary stringent hybridization conditions for hybridization of complementary nucleic acids which have more than 100 complementary residues on an array comprise 42oC using standard hybridization solutions (see, e.g., Sambrook), with the hybridization being carried out overnight. Exemplary highly stringent wash conditions can also comprise 0.15 M NaCl at 72oC for about 15 minutes. Exemplary stringent wash conditions can also comprise a 0.2×SSC wash at 65oC for 15 minutes (see, e.g., Sambrook). In one aspect, a high stringency wash is preceded by a medium or low stringency wash to remove background probe signal. An exemplary medium stringency wash for a duplex of, e.g., more than 100 nucleotides, comprises 1×SSC at 45oC for 15 minutes. An exemplary low stringency wash for a duplex of, e.g., more than 100 nucleotides, can comprise 4× to 6×SSC at 40oC for 15 minutes.

In alternative aspects, in making the arrays, and practicing the methods of the invention, the fluorescent dyes Cy3™ and Cy5™ can be used to differentially label nucleic acid fragments from two samples, e.g., nucleic acid generated from a control (e.g., “wild type”), versus a test cell or tissue sample, or, to label the array-immobilized nucleic acid and sample nucleic acid. Many commercial instruments are designed to accommodate the detection of these two dyes. To increase the stability of Cy5™, or fluors or other oxidation-sensitive compounds, antioxidants and free radical scavengers can be used in hybridization mixes, the hybridization and/or the wash solutions. Thus, Cy5™ signals are dramatically increased and longer hybridization times are possible.

In alternative aspects, the methods of the invention are carried out in a controlled, unsaturated humidity environment, and, the arrays of the invention can further comprise apparatus or devices capable of controlling humidity. Controlling humidity is one parameter that can be manipulated to increase hybridization sensitivity. Thus, in one aspect, in practicing the methods of the invention, hybridization can be carried out in a controlled, unsaturated humidity environment; hybridization efficiency is significantly improved if the humidity is not saturated. The hybridization efficiency can be improved if the humidity is dynamically controlled, i.e., if the humidity changes during hybridization. Array devices comprising housings and controls that allow the operator to control the humidity during pre-hybridization, hybridization, wash and/or detection stages can be used. The device can have detection, control and memory components to allow pre-programming of the humidity (and temperature and other parameters) during the entire procedural cycle, including pre-hybridization, hybridization, wash and detection steps.

In alternative aspects, the methods of the invention can incorporate hybridization conditions comprising temperature fluctuations and, the arrays of the invention can further comprise apparatus or devices capable of controlling temperature, e.g., an oven. Hybridization has much better efficiency in a changing temperature environment as compared to conditions where the temperature is set precisely or at relatively constant level (e.g., plus or minus a couple of degrees, as with most commercial ovens). Reaction chamber temperatures can be fluctuatingly modified by, e.g., an oven, or other device capable of creating changing temperatures.

In alternative aspects, the methods of the invention can comprise hybridization conditions comprising osmotic fluctuations, and, the arrays of the invention can further comprise apparatus or devices capable of controlling osmotic conditions, e.g., generate a e.g., a solute gradient. Hybridization efficiency (i.e., time to equilibrium) can also be enhanced by a hybridization environment that comprises changing hyper-/hypo-tonicity, e.g., a solute gradient. A solute gradient is created in a device. For example, a low salt hybridization solution is placed on one side of the array hybridization chamber and a higher salt buffer is placed on the other side to generate a solute gradient in the chamber.

Fragmentation and Digestion of Nucleic Acid

In practicing the methods of the invention, immobilized and sample nucleic acids can be cloned, labeled or immobilized in a variety of lengths. For example, in one aspect, the nucleic acid sequences comprising the same sequence or a sequence complementary to a sequence of a transcript expressed by a cell or an immobilized genomic nucleic acid segments can have a length smaller than about 200 bases. Use of labeled nucleic acid limited to this small size may improves the resolution of the molecular profile analysis, e.g., in array-based CGH. For example, use of such small fragments may allow for suppression of repetitive sequences and other unwanted, “background” cross-hybridization on the immobilized nucleic acid. Suppression of repetitive sequence hybridization greatly increases the reliability of the detection of copy number differences (e.g., amplifications or deletions) or detection of unique sequences.

The resultant fragment lengths can be modified by, e.g., treatment with DNase. Adjusting the ratio of DNase to DNA polymerase in a nick translation reaction changes the length of the digestion product. Standard nick translation kits typically generate 300 to 600 base pair fragments. If desired, the labeled nucleic acid can be further fragmented to segments below 200 bases, down to as low as about 25 to 30 bases, random enzymatic digestion of the DNA is carried out, using, e.g., a DNA endonucleases, e.g., DNase (see, e.g., Herrera (1994) J. Mol. Biol. 236:405-411; Suck (1994) J. Mol. Recognit. 7:65-70), or, the two-base restriction endonuclease CviJI (see, e.g., Fitzgerald (1992) Nucleic Acids Res. 20:3753-3762) and standard protocols, see, e.g., Sambrook, Ausubel, with or without other fragmentation procedures.

Other procedures can also be used to fragment nucleic acids, e.g., cDNA or genomic DNA, e.g. mechanical shearing, sonication (see, e.g., Deininger (1983) Anal. Biochem. 129:216-223), and the like (see, e.g., Sambrook, Ausubel, Tijssen). For example, one mechanical technique is based on point-sink hydrodynamics that result when a DNA sample is forced through a small hole by a syringe pump, see, e.g., Thorstenson (1998) Genome Res. 8:848-855. See also, Oefner (1996) Nucleic Acids Res. 24:3879-3886; Ordahl (1976) Nucleic Acids Res. 3:2985-2999. Fragment size can be evaluated by a variety of techniques, including, e.g., sizing electrophoresis, as by Siles (1997) J. Chromatogr. A. 771:319-329, that analyzed DNA fragmentation using a dynamic size-sieving polymer solution in a capillary electrophoresis. Fragment sizes can also be determined by, e.g., matrix-assisted laser desorption/ionization time-of-flight mass spectrometry, see, e.g., Chiu (2000) Nucleic Acids Res. 28:E31.

Comparative Genomic Hybridization (CGH)

In one aspect, methods of the invention incorporate array-based comparative genomic hybridization (CGH) reactions. CGH is a molecular cytogenetics approach that can be used to detect regions in a genome undergoing quantitative changes, e.g., gains or losses of sequence or copy numbers. In practicing the methods of the invention, CGH reactions can identify, locate (on the chromosome) and compare the genes encoding a cell's transcriptome, i.e., the transcripts expressed by one or more cells, e.g., a test versus a control cell population. For example, the methods of the invention can be used to identify what transcripts are being expressed and the physical location of the genes expressing them (i.e., gene mapping) in a genome. Practicing the methods of the invention can incorporate all known methods and means and variations thereof for carrying out comparative genomic hybridization, see, e.g., U.S. Pat. Nos. 6,197,501; 6,159,685; 5,976,790; 5,965,362; 5,856,097; 5,830,645; 5,721,098; 5,665,549; 5,635,351; and, Diago (2001) American J. of Pathol. May;158(5):1623-1631; Theillet (2001) Bull. Cancer 88:261-268; Werner (2001) Pharmacogenomics 2:25-36; Jain (2000) Pharmacogenomics 1:289-307.

Arrays, or “BioChips”

Making and using the arrays to practice the methods of the present invention can incorporate any known “array,” also referred to as a “microarray” or “DNA array” or “nucleic acid array” or “biochip,” or variation thereof. Arrays are generically a plurality of “target elements,” or “spots,” each target element comprising a defined amount of one or more biological molecules, e.g., polypeptides, nucleic acid molecules, or probes, immobilized on a defined location on a substrate surface. Typically, the immobilized biological molecules are contacted with a sample for specific binding, e.g., hybridization, between molecules in the sample and the array. Immobilized nucleic acids can contain sequences from specific messages (e.g., as cDNA libraries) or genes (e.g., genomic libraries), including, e.g., substantially all or a subsection of a chromosome or substantially all of a genome, including a human genome. Other target elements can contain reference sequences, such as positive and negative controls, and the like. The target elements of the arrays may be arranged on the substrate surface at different sizes and different densities. Different target elements of the arrays can have the same molecular species, but, at different amounts, densities, sizes, labeled or unlabeled, and the like. The target element sizes and densities will depend upon a number of factors, such as the nature of the label (the immobilized molecule can also be labeled), the substrate support (it is solid, semi-solid, fibrous, capillary or porous), and the like. Each target element may comprise substantially the same nucleic acid sequences, or, a mixture of nucleic acids of different lengths and/or sequences. Thus, for example, a target element may contain more than one copy of a cloned piece of DNA, and each copy may be broken into fragments of different lengths, as described herein. The length and complexity of the nucleic acid fixed onto the array surface is not critical to the invention. The array can comprise nucleic acids immobilized on any substrate, e.g., a solid surface (e.g., nitrocellulose, glass, quartz, fused silica, plastics and the like). See, e.g., U.S. Pat. No. 6,063,338 describing multi-well platforms comprising cycloolefin polymers if fluorescence is to be measured. Arrays used in the methods of the invention can comprise housing comprising components for controlling humidity and temperature during the hybridization and wash reactions.

In making and using the arrays and practicing the methods of the invention, known arrays and methods of making and using arrays can be incorporated in whole or in part, or variations thereof, as described, for example, in U.S. Pat. Nos. 6,323,043; 6,277,628; 6,277,489; 6,261,776; 6,258,606; 6,054,270; 6,048,695; 6,045,996; 6,022,963; 6,013,440; 5,973,708; 5,965,452; 5,959,098; 5,856,174; 5,830,645;5,770,456; 5,658,802; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765. The present invention can use any known array, e.g., SPECTRALCHIP™ Mouse BAC Arrays, SPECTRALCHIP™ Human BAC Arrays, G-CHIP™ and Custom Arrays of Spectral Genomics, Houston, Tex.; GENECHIPS™, Affymetrix, Santa Clara, Calif.; and, their accompanying manufacturer's instructions.

Substrate Surfaces

The arrays used to practice the methods of the invention can have substrate surfaces of a rigid, semi-rigid or flexible material. The substrate surface can be flat or planar, be shaped as wells, raised regions, etched trenches, pores, beads, filaments, or the like. Substrates can be of any material upon which a “capture probe” can be directly or indirectly bound. For example, suitable materials can include paper, glass (see, e.g., U.S. Pat. No. 5,843,767), ceramics, quartz or other crystalline substrates (e.g. gallium arsenide), metals, metalloids, polacryloylmorpholide, various plastics and plastic copolymers, Nylon™, Teflon™, polyethylene, polypropylene, poly(4-methylbutene), polystyrene, polystyrene/latex, polymethacrylate, poly(ethylene terephthalate), rayon, nylon, poly(vinyl butyrate), polyvinylidene difluoride (PVDF) (see, e.g., U.S. Pat. No. 6,024,872), silicones (see, e.g., U.S. Pat. No. 6,096,817), polyformaldehyde (see, e.g., U.S. Pat. Nos. 4,355,153; 4,652,613), cellulose (see, e.g., U.S. Pat. No. 5,068,269), cellulose acetate (see, e.g., U.S. Pat. No. 6,048,457), nitrocellulose, various membranes and gels (e.g., silica aerogels, see, e.g., U.S. Pat. No. 5,795,557), paramagnetic or superparamagnetic microparticles (see, e.g., U.S. Pat. No. 5,939,261) and the like. Reactive functional groups can be, e.g., hydroxyl, carboxyl, amino groups or the like. Silane (e.g., mono- and dihydroxyalkylsilanes, aminoalkyltrialkoxysilanes, 3-aminopropyl-triethoxysilane, 3-aminopropyltrimethoxysilane) can provide a hydroxyl functional group for reaction with an amine functional group.

Nucleic Acids and Detectable Moieties: Incorporating Labels and Scanning Arrays

In practicing the methods of the invention, nucleic acids associated with a detectable label are used. In one aspect, the invention provides a nucleic acid sequence comprising a detectable label, wherein the nucleic acid sequence comprises the same sequence or a sequence complementary to a sequence of a transcript expressed by a cell. The detectable label can be incorporated into, associated with or conjugated to the nucleic acid. Any detectable moiety can be used. The association with the detectable moiety can be covalent or non-covalent. In another aspect, the array-immobilized nucleic acids and sample nucleic acids are differentially detectable, e.g., they have different labels and emit difference signals.

Useful labels include, e.g., ³²P, ³⁵S, ³H, ¹⁴C, ¹²⁵I, ³I; fluorescent dyes (e.g., Cy5™, Cy3™, FITC, rhodamine, lanthanide phosphors, Texas red), electron-dense reagents (e.g. gold), enzymes, e.g., as commonly used in an ELISA (e.g., horseradish peroxidase, beta-galactosidase, luciferase, alkaline phosphatase), calorimetric labels (e.g. colloidal gold), magnetic labels (e.g. Dynabeads™), biotin, dioxigenin, or haptens and proteins for which antisera or monoclonal antibodies are available. The label can be directly incorporated into the nucleic acid to be detected, or it can be attached to a probe or antibody that hybridizes or binds to the target. A peptide can be made detectable by incorporating (e.g., into a nucleoside base) predetermined polypeptide epitopes recognized by a secondary reporter (e.g., leucine zipper pair sequences, binding sites for secondary antibodies, transcriptional activator polypeptide, metal binding domains, epitope tags). Label can be attached by spacer arms of various lengths to reduce potential steric hindrance or impact on other useful or desired properties. See, e.g., Mansfield (1995) Mol Cell Probes 9:145-156. In array-based CGH, fluors can be paired together; for example, one fluor labeling the control (e.g., the “nucleic acid of “known, or normal, karyotype”) and another fluor the test nucleic acid (e.g., from a chorionic villus sample or a cancer cell sample). Exemplary pairs are: rhodamine and fluorescein (see, e.g., DeRisi (1996) Nature Genetics 14:458-460); lissamine-conjugated nucleic acid analogs and fluorescein-conjugated nucleotide analogs (see, e.g., Shalon (1996) supra); Spectrum Red™ and Spectrum Green™ (Vysis, Downers Grove, Ill.); Cy3™ and Cy5™. Cy3™ and Cy5™ can be used together; both are fluorescent cyanine dyes produced by Amersham Life Sciences (Arlington Heights, Ill.). Cyanine and related dyes, such as merocyanine, styryl and oxonol dyes, are particularly strongly light-absorbing and highly luminescent, see, e.g., U.S. Pat. Nos. 4,337,063; 4,404,289; 6,048,982.

Other fluorescent nucleotide analogs can be used, see, e.g., Jameson (1997) Methods Enzymol. 278:363-390; Zhu (1994) Nucleic Acids Res. 22:3418-3422. U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

Detectable moieties can be incorporated into sample nucleic acid and, if desired, array-immobilized nucleic acid, by covalent or non-covalent means, e.g., by transcription, such as by random-primer labeling using Klenow polymerase, or “nick translation,” or, amplification, or equivalent. For example, in one aspect, a nucleoside base is conjugated to a detectable moiety, such as a fluorescent dye, e.g., Cy3™ or Cy5™, and then incorporated into a sample genomic nucleic acid. Samples of genomic DNA can be incorporated with Cy3™- or Cy5™-dCTP conjugates mixed with unlabeled dCTP. Cy5™ is typically excited by the 633 nm line of HeNe laser, and emission is collected at 680 nm. See also, e.g., Bartosiewicz (2000) Archives of Biochem. Biophysics 376:66-73; Schena (1996) Proc. Natl. Acad. Sci. USA 93:10614-10619; Pinkel (1998) Nature Genetics 20:207-211; Pollack (1999) Nature Genetics 23:41-46.

In another aspect, when using PCR or nick translation to label nucleic acids, modified nucleotides synthesized by coupling allylamine-dUTP to the succinimidyl-ester derivatives of the fluorescent dyes or haptenes (such as biotin or digoxigenin) are used; this method allows custom preparation of most common fluorescent nucleotides, see, e.g., Henegariu (2000) Nat. Biotechnol. 18:345-348.

Labeling with a detectable composition (labeling with a detectable moiety) also can include a nucleic acid attached to another biological molecule, such as a nucleic acid, e.g., a nucleic acid in the form of a stem-loop structure as a “molecular beacon” or an “aptamer beacon.” Molecular beacons as detectable moieties are well known in the art; for example, Sokol (1998) Proc. Natl. Acad. Sci. USA 95:11538-11543, synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (2001) Biochemistry 40:9387-9395, describing a molecular beacon comprised of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi (2001) Anal. Biochem. 294:126-131; Poddar (2001) Mol. Cell. Probes 15:161-167; Kaboev (2000) Nucleic Acids Res. 28:E94. Aptamer beacons can adopt two or more conformations, one of which allows ligand binding. A fluorescence-quenching pair is used to report changes in conformation induced by ligand binding. See also, e.g., Yamamoto (2000) Genes Cells 5:389-396; Smimov (2000) Biochemistry 39:1462-1468.

Detecting Dyes and Fluors

In addition to labeling nucleic acids with fluorescent dyes, the invention can be practiced using any apparatus or methods to detect “detectable labels” of a sample nucleic acid or an array-immobilized nucleic acid, or, any apparatus or methods to detect nucleic acids specifically hybridized to each other. In one aspect, devices and methods for the simultaneous detection of multiple fluorophores are used; they are well known in the art, see, e.g., U.S. Pat. Nos. 5,539,517; 6,049,380; 6,054,279; 6,055,325; 6,294,331. Any known device or method, or variation thereof, can be used or adapted to practice the methods of the invention, including array reading or “scanning” devices, such as scanning and analyzing multicolor fluorescence images; see, e.g., U.S. Pat. Nos. 6,294,331; 6,261,776; 6,252,664; 6,191,425; 6,143,495; 6,140,044; 6,066,459; 5,943,129; 5,922,617; 5,880,473; 5,846,708; 5,790,727; and, the patents cited in the discussion of arrays, herein. See also published U.S. patent applications Nos. 20010018514; 20010007747; published international patent applications Nos. WO0146467 A; WO9960163 A; WO0009650 A; WO0026412 A; WO0042222 A; WO0047600 A; WO0101144 A.

For example a spectrograph can image an emission spectrum onto a two-dimensional array of light detectors; a full spectrally resolved image of the array is thus obtained. Photophysics of the fluorophore, e.g., fluorescence quantum yield and photodestruction yield, and the sensitivity of the detector are read time parameters for an oligonucleotide array. With sufficient laser power and use of Cy5™ and/or Cy3™, which have lower photodestruction yields an array can be read in less than 5 seconds.

When using two or more fluors together (e.g., as in a CGH), such as Cy3™ and Cy5™, it is necessary to create a composite image of all the fluors. To acquire the two or more images, the array can be scanned either simultaneously or sequentially. Charge-coupled devices, or CCDs, are used in microarray scanning systems, including practicing the methods of the invention. Thus, CCDs used in the methods of the invention can scan and analyze multicolor fluorescence images. See, e.g., U.S. Pat. Nos. 5,552,827; 5,745,171; 5,852,468; 6,031,569; 6,323,901.

Color discrimination can also be based on 3-color CCD video images; these can be performed by measuring hue values. Hue values are introduced to specify colors numerically. Calculation is based on intensities of red, green and blue light (RGB) as recorded by the separate channels of the camera. The formulation used for transforming the RGB values into hue, however, simplifies the data and does not make reference to the true physical properties of light. Alternatively, spectral imaging can be used; it analyzes light as the intensity per wavelength, which is the only quantity by which to describe the color of light correctly. In addition, spectral imaging can provide spatial data, because it contains spectral information for every pixel in the image. Alternatively, a spectral image can be made using brightfield microscopy, see, e.g., U.S. Pat. No. 6,294,331.

Data Analysis

The methods of the invention further comprise data analysis, which can include the steps of determining, e.g., fluorescent intensity as a function of substrate position, removing “outliers” (data deviating from a predetermined statistical distribution), or calculating the relative binding affinity of the targets from the remaining data. The resulting data can be displayed as an image with color in each region varying according to the light emission or binding affinity between targets and probes. See, e.g., U.S. Pat. Nos. 5,324,633; 5,863,504; 6,045,996. The invention can also incorporate a device for detecting a labeled marker on a sample located on a support, see, e.g., U.S. Pat. No. 5,578,832.

Sources of Nucleic Acid

The invention provides methods of method for identifying a chromosomal loci of a gene expressed by a cell by hybridizing to an array a nucleic acid sequence comprising the same sequence or a sequence complementary to a sequence of a transcript expressed by a cell. In one aspect, the methods comprise use of samples of nucleic acids representing two different sources of transcripts, e.g., when performing an array-based comparative genomic hybridization (CGH).

The transcripts, i.e., the mRNA or messages, can be derived from (e.g., isolated from, amplified from, cloned from) any source. In one aspect, the cell, tissue or fluid sample from which the nucleic acid sample is prepared is taken from a patient suspected of having a pathology or a condition associated with abnormal polypeptide or transcript levels or genetic defects. The causality, diagnosis or prognosis of the pathology or condition may be associated with levels of transcript higher or lower than considered “normal” or wild type. The altered transcript levels may be due to gene amplification, increased or decreased transcription or an increase or decrease in message stability (e.g., decreased mRNA processing, increased or decreased message breakdown, and the like). The causality, diagnosis or prognosis of the pathology or condition may also be due to genetic defects, e.g., with genomic nucleic acid base substitutions, amplifications, deletions and/or translocations.

The cell, tissue or fluid can be from any source, e.g., amniotic samples, chorionic villus samples (CVS), serum, blood, chord blood or urine samples, CSF or bone marrow aspirations, fecal samples, saliva, tears, tissue and surgical biopsies, needle or punch biopsies, and the like.

Methods of isolating cell, tissue or fluid samples and message (mRNA) therefrom are well known to those of skill in the art and include, but are not limited to, aspirations, tissue sections, drawing of blood or other fluids, surgical or needle biopsies, and the like. A “clinical sample” derived from a patient includes frozen sections or paraffin sections taken for histological purposes. The sample can also be derived from supernatants (of cell cultures), lysates of cells, cells from tissue culture in which it may be desirable to identify a chromosomal loci of a gene expressed by a cell, including chromosomal abnormalities and copy numbers.

### EXAMPLES

The following example is offered to illustrate, but not to limit the claimed invention.

### Example 1

### Making Nucleic Acid Arrays

The following example demonstrates exemplary protocol for making an array of the invention.

Making BAC Microarrays:

BAC clones greater than fifty kilobases (50 kb), and up to about 300 kb, are grown up in Terrific Broth medium. Larger inserts, e.g., clones >300 kb, and smaller inserts, about 1 to 20 kb, are also be used. DNA is prepared by a modified alkaline lysis protocol (see, e.g., Sambrook). The DNA is labeled, as described below.

The DNA is then chemically modified as described by U.S. Pat. No. 6,048,695. The modified DNA is then dissolved in proper buffer and printed directly on clean glass surfaces as described by U.S. Pat. No. 6,048,695. Usually multiple spots are printed for each clone.

### Example 2

### Nucleic Acid Labeling and DNase Enzyme Fragmentation

A standard random priming method is used to label genomic DNA before its attachment to the array, see, e.g., Sambrook. Sample nucleic acid is also similarly labeled. Cy3™ or Cy5™ labeled nucleotides are supplemented together with corresponding unlabeled nucleotides at a molar ratio ranging from 0.0 to about 6 (unlabeled nucleotide to labeled nucleotides). Labeling is carried out at 37° C. for 2 to 10 hours. After labeling the reaction mix is heated up to 95° C. to 100° C. for 3 to 5 minutes to inactivate the polymerase and denature the newly generated, labeled “probe” nucleic acid from the template.

The heated sample is then chilled on ice for 5 minutes. “Calibrated” DNase (DNA endonuclease) enzyme is added to fragment the labeled template (generated by random priming). “Trace” amounts of DNase is added (final concentration was 0.2 to 2 ng/ml; incubation time 15 to 30 minutes) to digest/fragment the labeled nucleic acid to segments of about 30 to about 100 bases in size.

### Example 3

### Identifying a Chromosomal Loci of a Gene Expressed by a Cell

This example describes an array-based method for identifying a chromosomal loci of a gene differentially expressed by different cells by performing an array-based comparative genomic hybridization (CGH).

cDNA is prepared from cells of the germinal ridge of mice that are developmentally normal. This population of cDNAs is used as the reference (“normal” or “wild type”) sample. cDNA from the germinal ridge of mice at the same stage of development as the above is prepared. The mice are the same as those of the “reference” sample with the exception that these mice demonstrate phenotypic sex-reversal. Both samples are labeled, hybridized and analyzed using a G-CHIP™ (Spectral Genomics, Houston, Tex.) and using the manufacturer's protocol.

If a chromosomal loci is equally expressed by both cell populations, the methods of the invention will identify equitable hybridization by both samples (e.g., cDNA or fragments thereof of a transcript) to the array-immobilized genomic clones corresponding to the genes encoding the relevant transcripts. In addition to identifying the physical location of the gene encoding the transcript, the methods of the invention will identify if one locus or several loci are over-expressed or under-expressed by the test population. Additionally, the methods of the invention will identify if one locus or several loci are either expressed by the reference population but not by the test population or vice versa.

A number of embodiments of the invention have been described. Nevertheless, it will be understood that various modifications may be made without departing from the spirit and scope of the invention. Accordingly, other embodiments are within the scope of the following claims.

What is claimed is:

1. An array-based method for identifying a chromosomal loci of a gene expressed by a cell, the method comprising:
(a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome;
(b) providing a sample comprising a nucleic acid sequence comprising a detectable label, wherein the nucleic acid sequence comprises the same sequence or a sequence complementary to a sequence of a transcript expressed by a cell;
(c) contacting the sample of step (b) with the array of step (a) under conditions wherein the labeled nucleic acid can specifically hybridize to the genomic nucleic acid segments immobilized on the array; and,
(d) identifying to which discrete and known spots on the substrate surface are specifically hybridized to a labeled nucleic acid segment, wherein the identification is made by detecting a specifically hybridized nucleic acid sequence, thereby identifying a chromosomal loci of a gene expressed by a cell.

2. The method of claim 1, wherein the labeled nucleic acid sequence comprises the same sequence or a sequence complementary to a subset of transcripts expressed by the cell.

3. The method of claim 2, wherein the labeled nucleic acid sequence comprises the same sequence or a sequence complementary to all of the transcripts expressed by the cell.

4. The method of claim 1, wherein the transcript comprises a HER2/neu or a Neu/ErbB2 transcript.

5. The method of claim 1, wherein the transcript comprises a c-fos, a c-fas or a c-jun transcript.

6. The method of claim 1, wherein the transcript comprises a DAX-1 or a DAX-2 transcript.

7. An array-based method for identifying a chromosomal loci of a gene differentially expressed by different cells or by a cell under different conditions by performing an array-based comparative genomic hybridization (CGH), comprising the following steps:
(a) providing an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome;
(b) providing a first sample, wherein the sample comprises a plurality of nucleic acid sequences comprising a first detectable label, wherein the nucleic acid sequence comprises the same sequence or a sequence complementary to all of the transcripts expressed by a first cell or a subset of transcripts expressed by the first cell;
(c) providing a second sample, wherein the sample comprises a plurality of nucleic acid sequences comprising a second detectable label, wherein the nucleic acid sequence comprises the same sequence or a sequence complementary to all of the transcripts expressed by a second cell or a subset of transcripts expressed by the second cell;
(d) contacting the samples of step (b) and step (c) with the array of step (a) under conditions wherein the nucleic acid in the samples can specifically hybridize to the immobilized nucleic acid of step (a); and
(e) identifying to which discrete and known spots on the substrate surface are specifically hybridized to a nucleic acid sequence of step (b) and identifying to which discrete and known spots on the substrate surface are specifically hybridized to a nucleic acid sequence of step (c), wherein the identification is made by detecting a specifically hybridized first and a specifically hybridized second sample nucleic acid sequence, thereby identifying a chromosomal loci of a gene differentially expressed by a first cell compared to a second cell.

8. The method of claim 7, in step (e) further comprising measuring the amount of specifically hybridized first and second label on each discrete and known spot and comparing the amount of first and second sample nucleic acid sequence on each discrete and known spot, thereby determining the level of expression of the differentially expressed gene in the first cell as compared to the second cell.

9. The method of claim 7, wherein the first cell is a normal cell and the second cell has an abnormal phenotype.

10. The method of claim 9, wherein the abnormal phenotype comprises a disease phenotype.

11. The method of claim 9, wherein the abnormal phenotype comprises a neoplastic or hyperplastic phenotype.

12. The method of claim 11, wherein neoplastic phenotype is selected from the group consisting of breast cancer and bone cancer.

13. The method of claim 7, wherein the first cell is an unstimulated cell and the second cell is the unstimulated cell after stimulation.

14. The method of claim 7, wherein the first cell is an undifferentiated cell and the second cell is the undifferentiated cell after stimulation.

15. The method of claim 7, wherein the first cell is a normal cell and the second cell has phenotype of an injured cell.

16. The method of claim 7, wherein the first cell is a normal cell and the second cell has an altered phenotype because it has been exposed to an environmental stress.

17. The method of claim 16, wherein the environmental stress comprises a high or a low or a change in temperature.

18. The method of claim 16, wherein the environmental stress comprises an exposure to a chemical.

19. The method of claim 18, wherein the chemical is a carcinogen.

20. The method of claim 18, wherein the carcinogen is a drug or a medicine.

21. The method of claim 1 or claim 7, wherein the nucleic acid sequence comprises an RNA.

22. The method of claim 1 or claim 7, wherein the nucleic acid sequence comprises a DNA.

23. The method of claim 1 or claim 7, wherein the nucleic acid sequence comprises a cDNA.

24. The method of claim 1 or claim 7, wherein the nucleic acid sequence comprises an expressed sequence tag (EST).

25. The method of claim 1 or claim 7, wherein the nucleic acid sequence complementary to a transcript comprises a sequence representative of the full length of the transcript.

26. The method of claim 25, wherein the nucleic acid sequence complementary to a transcript is between about 12 to about 500 bases in length.

27. The method of claim 26, wherein the nucleic acid sequence complementary to a transcript is between about 25 to about 250 bases in length.

28. The method of claim 27, wherein the nucleic acid sequence complementary to a transcript is between about 50 to about 150 bases in length.

29. The method of claim 1, wherein the cloned genomic nucleic acid segment is cloned in a construct comprising an artificial chromosome.

30. The method of claim 29, wherein the artificial chromosome comprises a bacterial artificial chromosome (BAC).

31. The method of claim 29, wherein the artificial chromosome is selected from the group consisting of a human artificial chromosome (HAC) a yeast artificial chromosome (YAC), a transformation-competent artificial chromosome (TAC) and a bacteriophage P1-derived artificial chromosome (PAC).

32. The method of claim 1, wherein a cloned nucleic acid segment is cloned in a construct comprising a vector selected from the group consisting of a cosmid vector, a plasmid vector and a viral vector.

33. The method of claim 1, wherein the cloned nucleic acid segment is between about 50 kilobases (0.5 megabase) to about 500 kilobases (5 megabases) in length, between about 100 kilobases (1 megabase) to about 400 kilobases (4 megabases) in length, or, is about 300 kilobases (3 megabases) in length.

34. The method of claim 1, wherein the cell expressing the transcript of step (b) comprises a body fluid sample, a cell sample or a tissue sample.

35. The method of claim 1, wherein the cell expressing the transcript of step (b) comprises a cancer cell or a tumor cell sample.

36. The method of claim 1, wherein the cell expressing the transcript of step (b) comprises a biopsy sample.

37. The method of claim 1, wherein the cell expressing the transcript of step (b) comprises a blood sample.

38. The method of claim 1, wherein the cell expressing the transcript of step (b) comprises a urine sample.

39. The method of claim 1, wherein the cell expressing the transcript of step (b) comprises a cerebral spinal fluid (CSF) sample.

40. The method of claim 1, wherein the cell expressing the transcript of step (b) comprises an amniotic fluid sample.

41. The method of claim 1, wherein the cell expressing the transcript of step (b) comprises a chorionic villus sample.

42. The method of claim 1, wherein the cell expressing the transcript of step (b) comprises an embryonic cell or embryo tissue sample.

43. The method of claim 1, wherein the cell expressing the transcript of step (b) comprises a mammalian cell.

44. The method of claim 43, wherein the mammalian cell comprises a human cell.

45. The method of claim 1, further comprising a washing step, wherein nucleic acid in the sample not specifically hybridized to the genomic nucleic acid segments immobilized on the array are removed.

46. The method of claim 45, wherein the washing step comprises use of a solution comprising a salt concentration of about 0.02 molar at pH 7 at a temperature of at least about 50° C.

47. The method of claim 45, wherein the washing step comprises use of a solution comprising a salt concentration of about 0.15 M at a temperature of at least about 72° C. for about 15 minutes.

48. The method of claim 45, wherein the washing step comprises use of a solution comprising a salt concentration of about 0.2X SSC at a temperature of at least about 50° C. for at least about 15 minutes.

49. A kit comprising the following components:
(a) an array comprising a plurality of cloned genomic nucleic acid segments, wherein each genomic nucleic acid segment is immobilized to a discrete and known spot on a substrate surface to form an array and the cloned genomic nucleic acid segments comprise a substantially complete genome or a known subset of a genome; and,
(b) instructions for using the array for identifying a chromosomal loci of a gene expressed by a cell as set forth in claim 1 or claim 7.

50. The kit of claim 49, further comprising materials to prepare a sample comprising a genomic nucleic acid for application to the array.

51. The kit of claim 49, further comprising materials to isolate, clone or amplify the transcripts of a cell.

52. The kit of claim 49, further comprising materials to prepare a cDNA library.

53. The kit of claim 49, further comprising materials to label a sample.

54. The kit of claim 49, further comprising an array-immobilized nucleic acid.

55. The kit of claim 49, further comprising a sample of wild type, or normal, nucleic acid.

56. The kit of claim 55, wherein the wild type, or normal, nucleic acid is labeled.

57. The kit of claim 56, wherein the wild type, or normal, nucleic acid comprises a human wild type genomic nucleic acid.

58. The kit of claim 54, wherein the array comprises a G-CHIP™, a SPECTRALCHIP™ Mouse BAC Array or a SPECTRALCHIP™ Human BAC Array.
